(19) United States
(12) Patent Application Publication
(10) Pub. No.: US 2005/0164188 A1
(43) Pub. Date: Jul. 28, 2005

(54) Analysis device

(76) Inventor: Jeffrey Kane, Chelsea, MI (US)

Correspondence Address: LEYDIG VOIT & MAYER, LTD, 700 THIRTEENTH ST. NW, SUITE 300, WASHINGTON, DC, 20005-3960, US

(21) Appl. No.: 10/501,881

(22) PCT Filed: Jan. 24, 2003
(86) PCT No.: PCT/US03/01942

Related U.S. Application Data
(60) Provisional application No. 60/350,376, filed on Jan. 24, 2002.

(51) Int. Cl. C12Q 1/68; C12M 1/34

(52) U.S. Cl. 435/6; 435/287.2

(57) ABSTRACT

An analysis device comprising a microporous membrane integrally bonded to a non-porous polymeric injection-molded support is disclosed. Methods for making and using the analysis device are also provided.

### CROSS-REFERENCE TO RELATED PATENT APPLICATIONS

This patent application claims the benefit of U.S. Provisional Patent Application No. 60/350,376, filed Jan. 24, 2002, which is incorporated by reference.

### FIELD OF THE INVENTION

This invention relates to analysis devices comprising membranes integrally bonded to non-porous polymeric supports.

### BACKGROUND OF THE INVENTION

Many techniques for analyzing biomolecules, such as nucleic acids or proteins, include placing a sample containing the biomolecules on a solid surface such as a supported membrane, or a non-porous glass or polymeric slide, and placing a binding agent, such as a complementary nucleic acid probe or antibody that will specifically bind to the biomolecule, in contact with the biomolecules and forming a complex between the biomolecule and binding agent. Alternatively, and more typically, a binding agent can be immobilized on the solid surface and the biomolecule in the sample can be placed in contact with the binding agent to form a complex. The binding agent may be labeled before use and/or one or more labels may be added to the complex. Depending on the particular technique, additional binding agents (e.g., nucleic acid probes or anti-antibodies) and/or labeling reagents can also be utilized. The label that is bound to the complex is subsequently detected, thus indicating the presence of the biomolecules of interest.

Analysis devices have been developed wherein small volumes of fluid e.g., containing the sample or a binding agent are deposited (typically by printing or ink jetting) at predetermined locations on the solid surface, and the other member(s) of the complex is subsequently added to the location in a similar manner so the complex can be formed. Some devices allow the binding agent to be synthesized on the surface before the sample is added. These devices, having material deposited in a microarray pattern, allow numerous samples to be analyzed simultaneously, and are particularly suitable for automated analysis.

However, analysis devices have suffered from a number of drawbacks. For example, some solid surfaces have exhibited insufficient and/or inconsistent binding capacity or binding efficiency. Additionally or alternatively, some devices, e.g., including membranes attached to glass supports via adhesives, have been labor intensive to produce, difficult to handle and/or are not particularly suitable for automated analysis.

The present invention provides for ameliorating at least some of the disadvantages of the prior art. These and other advantages of the present invention will be apparent from the description as set forth below.

### BRIEF SUMMARY OF THE INVENTION

In accordance with an embodiment of the invention, an analysis device is provided comprising a microporous membrane integrally bonded to a non-porous polymeric injection-molded support. A preferred embodiment of the analysis device comprises a microporous membrane having a thickness reduced by at least about ten percent when compared to the thickness of the microporous membrane before bonding it to the support. In a more preferred embodiment of the analysis device, the membrane also has a pore structure reduced by at least about ten percent when compared to the pore structure of the microporous membrane before bonding it to the support. The membrane is suitable for binding biomolecules such as nucleic acids, proteins, and antibodies, and the analysis device has a variety of applications, especially in hybridization assays and immunoassays.

Embodiments of the invention are particularly useful as microarray devices, e.g., wherein samples containing nucleic acids to be tested or evaluated, and nucleic acids containing nucleotide sequences complementary to those of the nucleic acids to be tested or evaluated, are deposited in a microarray pattern on a first surface of the membrane, and complexes formed between the complementary sequences are detected. Even more preferably, the microarray devices can be used in automated protocols, e.g., they are compatible with conventional scanning and analysis equipment.

In accordance with other embodiments of the invention, methods for making the analysis device are also provided.

### BRIEF DESCRIPTION OF THE DRAWINGS

FIG. 1 is a diagrammatic top view of an embodiment of the analysis device of the present invention showing a microporous membrane integrally bonded to a non-porous polymeric injection-molded support.

FIG. 2 is a diagrammatic side view of an embodiment of the analysis device of the present invention, wherein a surface of the support has a raised portion contacting a surface of the membrane.

FIG. 3 is a diagrammatic side view of another embodiment of the analysis device of the present invention, wherein a surface of the support does not have a raised portion contacting a surface of the membrane.

FIG. 4 is a diagrammatic cross-sectional view of embodiments of empty molds for forming an analysis device of the present invention, showing a core half (the membrane-receiving portion of the mold) and a cavity half (the polymer-injecting portion of the mold). FIG. 4a shows a core half and a cavity half, and FIG. 4b shows another embodiment of a cavity half.

FIG. 5 is a magnified top view of a nylon membrane before being integrally bonded to the support (FIG. 5a, magnification 4000×, 0.2μ average pore size) and after being integrally bonded to the support (FIG. 5b; magnification 4000×), showing the reduction in pore structure in the bonded membrane compared to the non-bonded membrane.

FIG. 6 is a magnified cross-sectional view of a nylon membrane before being integrally bonded to the support (FIG. 6a, magnification 860×, 0.2μ average pore size, the thickness of the membrane shown by the arrows) and after being integrally bonded to the support (FIG. 6b, magnification 1000×, the thickness of the membrane shown by the arrows), showing the reduction in thickness, voids volume, and pore structure in the bonded membrane compared to the non-bonded membrane.

### DETAILED DESCRIPTION OF THE INVENTION

In accordance with an embodiment of the invention, an analysis device comprises a microporous membrane integrally bonded to a non-porous polymeric injection-molded support. In a more preferred embodiment, the thickness of the bonded membrane is reduced as compared to the thickness of the membrane before the integral bond is formed, e.g., the heat and pressure of injection molding compresses the membrane while providing an integral bond between a surface of the membrane and a surface of the support. For example, in one embodiment of the analysis device, the microporous membrane has a first surface and a second surface, and a bulk disposed between the first and second surfaces, the bulk having a thickness, wherein the bulk thickness of the membrane is reduced (preferably by at least about 10%) by the heat and pressure of injection molding when compared to the bulk thickness of the membrane before the injection-molded support is formed. In some embodiments, a surface of the support has a raised portion, and the raised portion is integrally bonded to a surface of the membrane.

Another embodiment of an analysis device comprises a microporous membrane integrally bonded to a non-porous polymeric injection-molded support, wherein the microporous membrane comprises a polymeric membrane having a pore structure (e.g., an average pore size, a nominal pore size, or an average pore diameter) that is reduced, preferably by at least about ten percent, when compared to the pore structure of the microporous polymeric membrane before bonding it to the support.

Without being bound to any particular theory, it is believed the compressed membrane, e.g., having a reduced thickness and pore structure, reduces the diffusion of the sample and the binding agent(s), while maintaining sufficient binding capacity, thus optimizing analysis.

An analysis device provided by another embodiment of the invention comprises a microporous membrane integrally bonded to a non-porous polymeric support by injection molding.

While the analysis device can have any suitable dimensions, in one embodiment, the analysis device has the general dimensions of a standard microscope slide. Preferably, the device is compatible with automated analysis equipment. Additionally, analysis devices according to the present invention are simple to manufacture and are especially suited to automated fabrication.

In accordance with an embodiment of the invention, a method for making an analysis device comprises placing a membrane having a first surface and a second surface in a mold core half, placing the mold core half in contact with a mold cavity half, injecting a polymer into the mold cavity half such that the polymer contacts the second surface of the membrane, and forming an analysis device comprising a microporous membrane having a first surface and a second surface and a non-porous injection-molded support having a first surface and a second surface, wherein the second surface of the membrane is integrally bonded to the second surface of the support.

In another embodiment, an analysis device is produced by the process comprising positioning a first surface of a microporous membrane adjacent a surface of an injection mold and injecting a polymer into the injection mold to form an injection molded support and integrally bond a second surface of the microporous membrane to the injection molded support. Without being bound to any particular mechanism, it is believed polymer injected into the mold flows into at least some of the pores of the microporous membrane and integrally bonds the membrane to the injection-molded support.

In yet another embodiment, a method of bonding a microporous membrane to a non-porous polymeric support is provided, comprising positioning a first surface of a microporous membrane adjacent a surface of an injection mold, and injecting a polymer into the injection mold to form an injection molded support integrally bonded to a second surface of the microporous membrane.

Embodiments of analysis devices according to the invention have a variety of applications, especially in hybridization assays and immunoassays, including, but not limited to, arrays (e.g., microarrays, including but not limited to those described in Friend et al., Scientific American, February: 44-53 (2002), Marshall et al., Nature Biotechnology, 16: 27-31 (1998), and Schena, M. et al., Trends in Biotechnology, 16: 301-306 (1998)). Embodiments of the invention are compatible with automated and semi-automated protocols, as well as high throughput applications, and are especially suitable for bioinformatics applications, e.g., for data mining and data visualization.

In preferred embodiments of hybridization assays according to the invention (that can include, for example, mRNA abundance analyses, determining the presence and/or sequence of genes, monitoring levels of gene expression, and determining the presence or absence of known or new mutations in gene sequences, e.g., SNP genotyping), numerous probes and samples are deposited in a microarray pattern on a single analysis device, and the complexes are subsequently detected essentially simultaneously, e.g., via automated analytical protocols.

A hybridization method for analyzing biomolecules according to an embodiment of the invention comprises providing (e.g., depositing) at least one binding agent comprising one or more probe nucleic acids having nucleotide sequences on a first surface of a microporous membrane of an analysis device such that the one or more probe nucleic acids are immobilized, the probe nucleic acid nucleotide sequences being complementary to a nucleotide sequence of at least one biomolecule of interest, the analysis device comprising the microporous membrane integrally bonded to a non-porous polymeric injection-molded support, the membrane having a first surface for receiving the probe nucleic acids and a sample containing the biomolecule, the membrane having a second surface integrally bonded to a surface of the support; depositing at least one sample containing the biomolecule onto the first surface of the membrane such that the biomolecule contacts the probe nucleic acid and a complex is formed between the probe nucleic acid nucleotide sequence and the complementary nucleotide sequence of the biomolecule; and, detecting the complex. Preferably, a plurality of probe nucleic acids and a plurality of biomolecules of interest are deposited onto the first surface of the membrane, and a plurality of complexes are formed and subsequently detected.

In another embodiment of the method, the at least one sample containing the at least one biomolecule of interest is initially placed on the first surface of the membrane and immobilized, and one or more probes are subsequently deposited on the membrane such that one or more complexes are formed, and subsequently detected.

Other embodiments of the invention include, for example, immunoassays, e.g., wherein a binding agent comprising at least one antibody is initially deposited on the first surface, and at least one sample is subsequently deposited to form a complex, or wherein at least one sample is initially deposited, and a binding agent comprising an antibody is subsequently deposited to form a complex. Preferably, a plurality of biomolecules and binding agents are deposited, and a plurality of complexes are formed and subsequently detected.

In accordance with any assay embodiment of the invention, additional reagents, e.g., one or more binding agents (including, for example, anti-antibodies and/or specific binding agents), or labels, can be utilized, e.g., to form a complex, or to label a complex. A variety of labels (e.g., radioactive, fluorescent, or chemiluminescent), including a plurality of distinguishable labels when two or more labels are used in an assay, can be utilized as is known in the art.

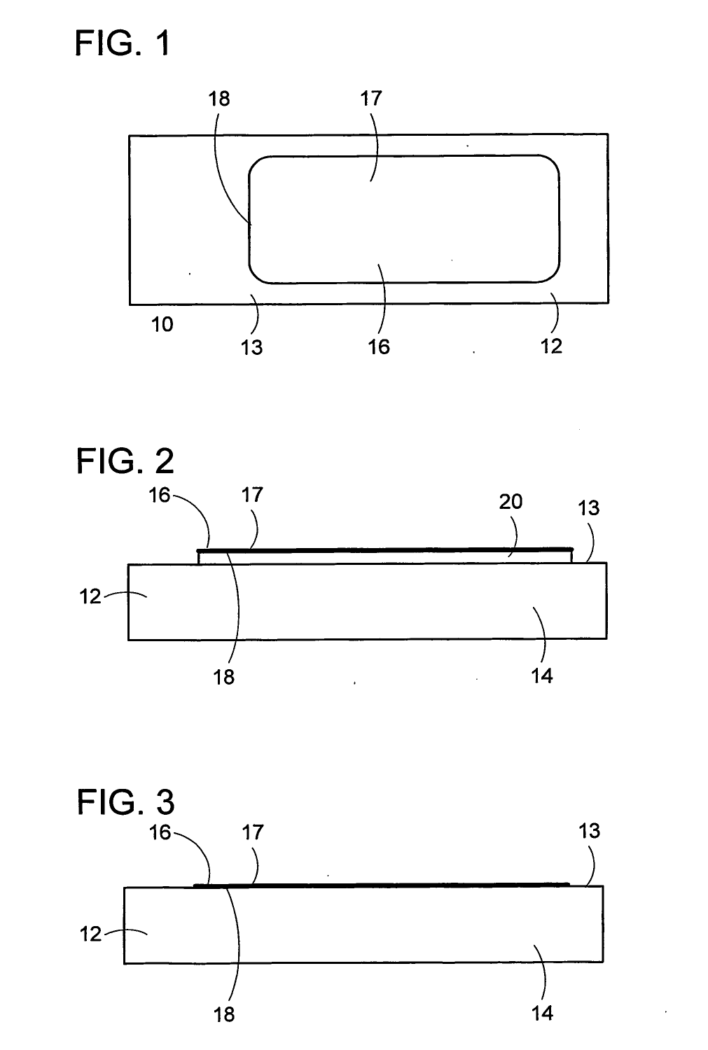

A preferred embodiment of an analysis device according to the present invention is illustrated in FIG. 1. The analysis device 10 includes a polymeric non-porous injection-molded support 12 having a first surface 13 and a second surface 14, and a microporous membrane 16 for receiving the binding agent(s) and biomolecules, the membrane 16 having a first surface 17 and a second surface 18, wherein the microporous membrane 16 is integrally bonded to the polymeric support 12.

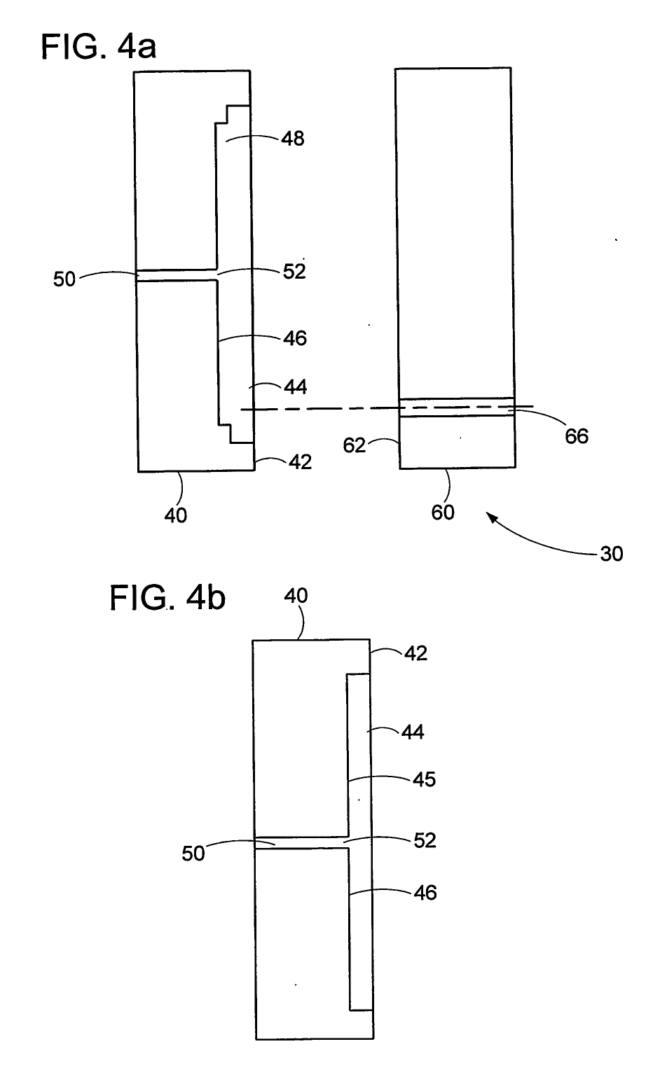

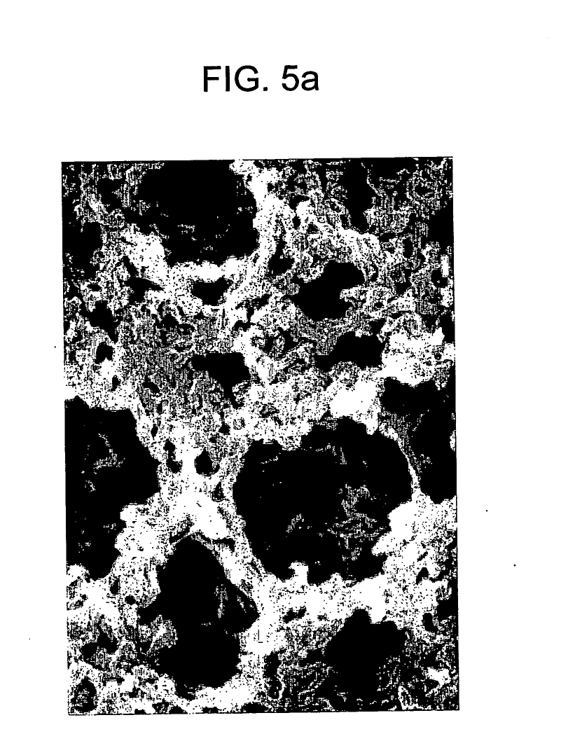

In an embodiment of an analysis device according to the present invention, best illustrated in FIG. 2, the polymeric support 12 includes a raised portion or “step” 20, wherein the microporous membrane 16 is integrally bonded thereto. In another embodiment, best illustrated in FIG. 3, the polymeric support 12 does not include a raised portion.

As used herein, “integrally bonded” means (using FIGS. 1 and 3 for reference) a surface of the membrane, surface 18, is bound to a surface of the support, surface 13, without a separate adhesive or adhesive layer interposed between the membrane 16 and the support 12.

In a variation of the embodiments shown in FIGS. 1-3, the microporous membrane comprises a composite, e.g., the membrane comprises a membrane layer that receives the deposited binding agent(s) and biomolecules, and an additional layer such as an additional membrane or a film bound thereto, wherein a surface of the additional membrane layer or film layer is integrally bonded to a surface of the polymeric non-porous injection molded support.

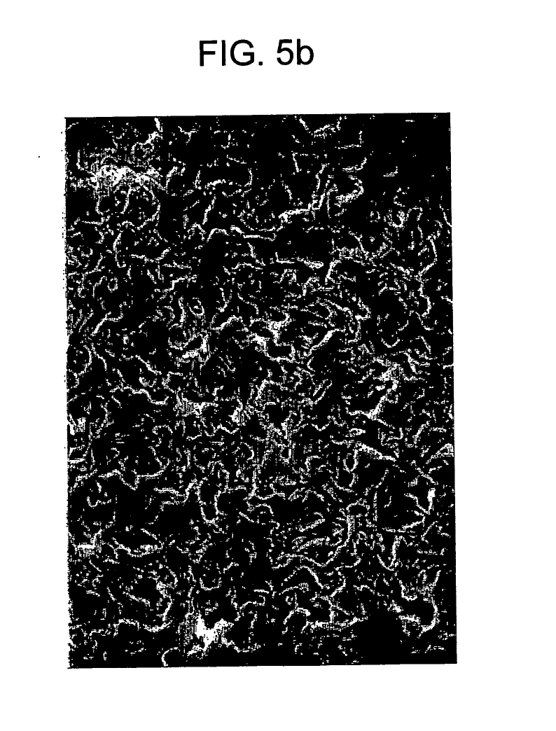

A diagrammatic cross-sectional view of an embodiment of a mold 30 comprising a mold cavity half 60 and a mold core half 40 for forming an analysis device according to the present invention is illustrated in FIG. 4a, and FIG. 4b shows another embodiment of a mold core half 40. The mold cavity half 60, through which the polymer is injected, and the mold core half 40, which receives the membrane and the injected polymer, are cooperatively configured to contact each other and form a mold cavity therebetween. In accordance with a typical embodiment of preparing an analysis device, the mold cavity half remains stationary, and the mold core half is movable, i.e., the core half is placed in contact with the mold cavity to close the mold, and moved away from the mold cavity to open the mold. The mold core and mold cavity halves can be oriented and operated as is known in the art, e.g., vertically, or horizontally. Additionally, the membrane can be retained in the mold core half as is known in the art, e.g., using vacuum, gravity, or retractable pins.

In accordance with the embodiment illustrated in FIG. 4a, the mold cavity formed by the mold cavity half 60 and the mold core half 40 has a substantially rectangular shape and thickness corresponding to the support 12 of analysis device 10 shown in FIG. 2. However, the mold cavity may have any desired configuration in accordance with the desired configuration of the polymeric support. For example, when the mold cavity has the configuration formed by mold core half 40 in FIG. 4b and mold core cavity half 60 in FIG. 4a, the configuration corresponds to the support 12 of analysis device 10 shown in FIG. 3.

The membrane-retaining section of the mold core half 40 typically includes a mold face 42, a polymer-receiving cavity 44, and a membrane-receiving surface 46. The membrane-retaining section of the illustrated embodiment of the mold core half 40 also includes a vacuum channel 50, and at least one port or opening 52 communicating with the vacuum channel 50 and a vacuum source (not shown). In the illustrated embodiment, the membrane-receiving surface 46 includes the opening 52.

In the embodiment illustrated in FIG. 4a, the membrane-receiving surface 46 is formed in a pocket or depression 48 in the mold core half 40. Preferably, the pocket 48 corresponds generally to the shape of the microporous membrane, although the pocket 48 may alternatively have other configurations. The pocket 48 when filled with polymer forms a “step” on the support, for example, corresponding to the step 20 seen in the analysis device illustrated in FIG. 2.

Alternatively, as shown in FIG. 4b, the mold core half 40 may not include a pocket 48, thus providing, for example, the analysis device support 12 illustrated in FIG. 3. In those embodiments, the membrane-receiving surface 46 may comprise a substantially flat region in a rear-wall 45 of the polymer-receiving cavity 44.

The polymer-injecting section of the mold cavity half 60 preferably includes a mold face 62 including at least one opening, channel, or nozzle 66 through which polymer is injected into the mold 30. The opening 66 may be configured to sealingly engage a nozzle or may have any suitable configuration for injecting a polymer.

In other embodiments, the mold cavity half 60 and/or mold core half 40 can be configured to provide, for example, a support with an indented portion on the lower surface (e.g., to avoid scratches) and/or a support with finger indents for ease in handling.

In operation, the first surface 17 of the microporous membrane 16 is positioned, either manually or automatically, against the membrane-receiving surface 46 of the mold core half 40. In accordance with this illustrated embodiment, the membrane is held in place by the suction generated by the vacuum source. The mold core half 40 is moved toward the mold cavity half 60 so that the core half face 42 and the cavity half face 62 are in contact. The mold halves are held tightly together forming a fluid-tight mold cavity.

Molten polymer is injected into the mold cavity at an elevated pressure, filling the portion of the mold core half 40 (FIG. 4a, also including the pocket or depression 48; and FIG. 4b) adjacent the microporous membrane 16, forming a support 12 having the shape of the mold cavity 32, and integrally bonding the second surface 18 of the microporous membrane 16 to the first surface 13 of the support 12. The mold core half 40 is subsequently moved away from the mold cavity half 60 such that the mold is opened, and the formed analysis device is removed. The injection molding process is carried out as is known in the art, and the selection of the appropriate conditions for the polymer, e.g., temperature and pressure, are matters of routine choice to the ordinary artisan.

As noted above, in some embodiments, the mold core half 40 does not include a vacuum chamber. For example, the microporous membrane can be held in place by one or more retractable pins (not shown). Alternatively, the microporous membrane can be held in place by gravity.

In some embodiments wherein the membrane comprises a composite, e.g., the membrane comprises a membrane layer that receives the deposited binding agent(s) and biomolecule(s), and an additional layer such as an additional membrane or a film bound thereto, the additional layer (that can be more thermally resistant, and can comprise a different polymer than the membrane receiving the deposited material) provides thermal insulation between the membrane layer (for receiving the binding agent(s) and biomolecule(s)) and the molten polymer while the analysis device is being formed. Such an arrangement can be desirable for those embodiments wherein the membrane layer for receiving the binding agent and biomolecules may otherwise be affected by the temperatures utilized during injection molding. Accordingly, during the formation of the analysis device, the composite membrane is arranged in the mold core half 40 such that the molten polymer contacts the additional layer (rather than the membrane layer for receiving the binding agent and biomolecules) resulting in an integral bond between the additional layer and the injection-molded support.

During injection molding and while forming the integral bond, the microporous membrane (or the microporous membrane layer for receiving the binding agent and biomolecules) is compressed, reducing the membrane thickness. Without being limited to any particular mechanism, it is believed that the temperature and pressure at which the polymer is injected bonds the membrane to the support and compresses the microporous membrane. For example, the microporous membrane thickness is preferably reduced by at least about 10%, more preferably by at least about 20%. In some embodiments, the membrane thickness is reduced by at least about 30%, or by at least about 50%. Compressing the membrane not only reduces the thickness, but can also reduce the membrane pore structure (e.g., the average pore size, the nominal pore size, or the average pore diameter). For example, the pore structure (e.g., the average pore size) of the microporous membrane is preferably reduced by at least about 10%, more preferably by at least about 20%. In some embodiments, the average pore size is reduced by at least about 30%, or by at least about 50%. In some embodiments, the average pore size is reduced by 75%, or more. Alternatively, or additionally, the void volume of the membrane can also be reduced, e.g., by about 10% or more, in some embodiments, at least about 20% or more.

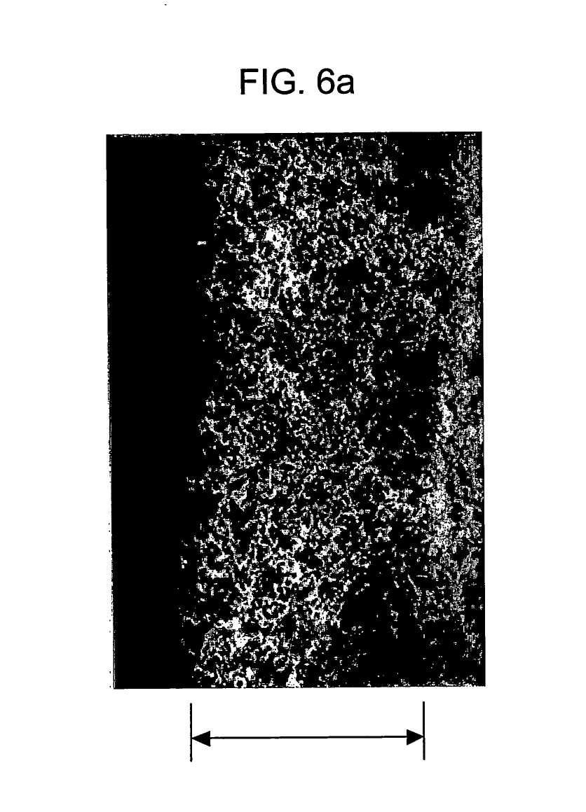

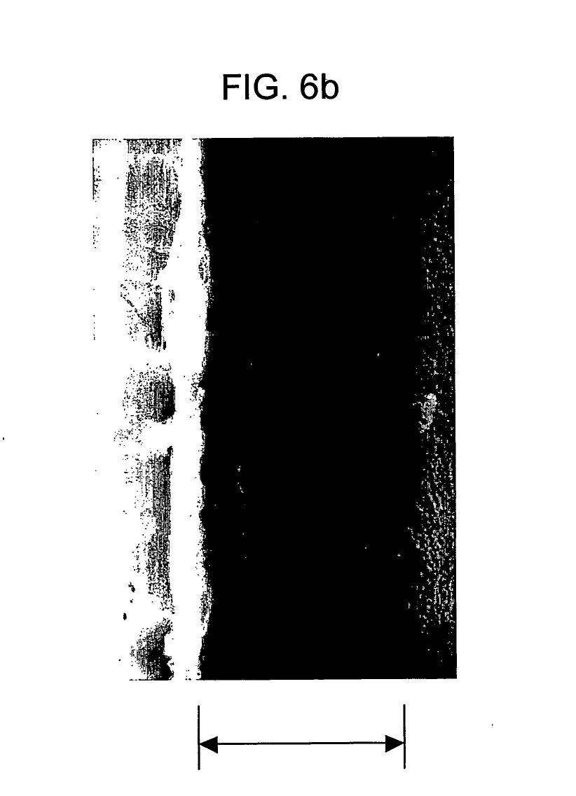

FIGS. 5 and 6 show an embodiment wherein the thickness and the average pore size of the membrane is reduced by at least about 50% after forming the integral bond (FIGS. 5b and 6b).

A variety of membranes, preferably microporous polymer membranes, are suitable for use in the invention. Examples of suitable polymers include polyaromatics, sulfones (including polysulfones such as aromatic polysulfones, for example, polyethersulfone, bisphenol A polysulfone, polyarylsulfone, and polyphenylsulfone), polyolefins, polystyrenes, polycarbonates, polyamides (e.g., nylon, including nylon 6, 6T, 11, 46, 66, and 610), polyimides, polyvinylidene fluoride, fluoropolymers, cellulosic polymers such as cellulose acetates and cellulose nitrates, and PEEK. Polyethersulfone and nylon are particularly preferred.

Suitable membranes can be unmodified or modified to include a surface charge, e.g., a positive or negative charge, or to alter the polarity or hydrophilicity of the surface. Examples of such modifications include grafting, e.g., irradiation, a polar or charged monomer, coating and/or curing the surface with a charged polymer, and carrying out conventional chemical modification to attach functional groups on the surface. If desired, the membranes can be suitable for binding the biomolecules through covalent interaction, or non-covalent bonds, e.g., hydrophobic and/or ionic attraction.

The porous membrane can have any suitable pore structure (before or after compression). For example, the membrane can have an average pore size of below about 10 μm. Typically, before compression, the membrane has an average pore size in the range of from about 0.01 μm to about 10 μm, preferably from about 0.1 μm to about 5 μm, and more preferably from about 0.2 μm to about 5 μm. The membranes can be, for example, asymmetric or symmetric membranes.

The porous membrane can have any suitable thickness (before or after compression). For example, the membrane can have a thickness in the range of from about 1 μm to about 25 μm. Typically, before compression, the membrane has a thickness of from about 4 μm to about 10 μm, and more preferably from about 4 μm to about 6 μm.

Suitable membranes include, but are not limited to, those described in U.S. Pat. Nos. 4,340,479, 4,702,840, 4,707,266, 4,900,449, 4,906,374, 4,964,989, 4,964,990, 5,108,607, 5,277,812 and 5,531,893, and International Publication No. WO 98/21588.

Suitable commercially available membranes include, but are not limited to, those available from Pall Corporation (East Hills, N.Y.) under the trade names BIODYNE® PLUS, BIODYNE® A, BIODYNE® B, BIODYNE® C, POSIDYNE®, LOPRODYNE® LP, SUPOR®, SUPOR® 30Q, SUPOR® 30 PLUS, PREDATOR®, ULTRABIND™, MUSTANG®, and IMMUNODYNE® ABC.

Any polymer suitable for injection molding and for forming a substantially rigid, non-porous support may be used in accordance with the present invention. A variety of polymers are known and are commercially available. Suitable polymers include, but are not limited to, polystyrene, polyolefin, polycarbonate, polyvinyl chloride, polyurethane, and acrylic. The polymer can be selected to provide a transparent or opaque support. Alternatively, or additionally, the support can be dyed or coated to provide any desired color.

If desired, the formed devices can be packaged, e.g., individually, or in packs of multiple devices. In some embodiments, the analysis devices include bar-coding, e.g., for ease in data tracking.

Once the analysis device is formed, samples (e.g., containing one or more biomolecules of interest) and reagents (e.g., binding agents and labels) can be applied to the device, and various assays can be performed, as is known in the art.

As used herein, the term biomolecules includes, but is not limited to, nucleic acid sequences, e.g., natural or synthetic DNA (for example, cDNA obtained after transcribing mRNA), RNA (including mRNA), and/or PNA (peptide nucleic acids); mixtures and/or hybrids thereof, as well as oligonucleotides, modified nucleic acids, fragments and/or derivatives of nucleic acids), antigens, proteins (including antibodies, and some antigens), peptides, bacteria, viruses, protozoans (as well as components of bacteria, viruses, and protozoans), and one or more analytes of interest (e.g., recombinant nucleic acid products and/or byproducts, drugs, pollutants, and poisons).

The biomolecules can be obtained from a variety of sources. A sample containing the biomolecules can comprise one or more cells, or an aqueous or aqueous miscible solution that is obtained directly from a liquid source or as a wash from a solid material, a growth medium or buffer solution in which biomolecules are present or have been introduced. In some embodiments, the sample is obtained from a biological fluid, including separated or unfiltered fluids such as blood or blood components, urine, cerebrospinal fluid, lymph fluids, tissue homogenate, cell extracts, saliva, sputum, stool, or physiological secretions. The sample can be obtained from an environmental source, e.g., a waste stream, a water source, a supply line, or a production lot. Industrial sources include fermentation media, such as from a biological reactor or food fermentation process such as brewing, or foodstuffs, such as meat, produce, or dairy products.

As used herein, the term “binding agent” includes, but is not limited to, one or more ligands and receptors, e.g., nucleic acid probes and/or antibodies (including a monoclonal antibodies, polyclonal antibodies, and anti-antibodies). Preferably at least one binding agent, e.g., the binding agent that is capable of binding to the biomolecules of interest, is a specific binding agent. For example, in some embodiments wherein the biomolecules of interest is a nucleic acid sequence, the specific binding agent has at least about 30% (more preferably, at least about 50%) complementarity with a region in the biomolecules of interest. The desired specificity can vary, depending on the particular nature of the biomolecules to be detected, the information desired about the nature of the sample, and the like.

Typically, in those embodiments including a plurality of binding agents, e.g., a first binding agent and a second binding agent, the first binding agent is capable of binding to the biomolecules, and the second binding agent is capable of binding to either the first binding agent (e.g., as in a double antibody assay), or to the biomolecules (e.g., as in a sandwich assay).

In accordance with the invention, detecting the biomolecules can include confirming the presence of the biomolecules, as well as (if desired) identifying the biomolecules, analyzing the biomolecules, and/or quantifying the biomolecules. Biomolecules can be detected as part of an on-going or monitoring process, e.g., as part of a quality control system, and/or to monitor the appearance/removal (or rate of appearance/removal) of the biomolecules in the sample, in the material of interest and/or in the product or process fluid being produced.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. An analysis device comprising:
a microporous polymeric membrane integrally bonded to a non-porous injection-molded polymeric support.

2. The analysis device of claim 1, wherein the support includes a raised portion, and the membrane is integrally bonded to the raised portion.

3. The analysis device according to claim 1, wherein the membrane has a first and second surface and a bulk disposed between the first and second surface, the bulk having a thickness and wherein the bulk thickness is reduced by the heat and pressure of injection molding when the polymeric support is formed.

4. The analysis device according to claim 1, wherein the bulk thickness of the membrane is reduced by at least about ten percent when compared to the bulk thickness of the membrane before bonding it to the support.

5. The analysis device according to claim 1 comprising a membrane having a pore structure reduced by at least about ten percent when compared to the pore structure of the membrane before bonding it to the support.

6. The analysis device of claim 5, comprising a membrane having an average pore size reduced by at least about ten percent when compared to the average pore size of the membrane before bonding it to the support.

7. The analysis device of claim 1, wherein the membrane comprises a sulfone membrane.

8. The analysis device of claim 1 wherein the membrane comprises a polyamide membrane.

9. The analysis device of claim 7, wherein the membrane is a polyethersulfone membrane.

10. The analysis device of claim 8, wherein the membrane is a nylon membrane.

11. The analysis device of claim 1, wherein the membrane comprises a composite including 2 layers.

12. The analysis device of claim 1, wherein the support comprises a polymer selected from the group consisting of polystyrene, polyolefin, polycarbonate, polyvinyl chloride, polyurethane, and acrylic.

13. An analysis device comprising a microporous membrane integrally bonded to a non-porous polymeric support by injection molding.

14. A method for making an analysis device comprising:
placing a membrane having a first surface and a second surface in a mold core half;
placing the mold core half in contact with a mold cavity half;
injecting a polymer into the mold cavity half such that the polymer contacts the second surface of the membrane, and forming an analysis device comprising a microporous membrane having a first surface and a second surface and a non-porous injection-molded support having a first surface and a second surface, wherein the second surface of the membrane is integrally bonded to the second surface of the support.

15. The method of claim 14, wherein the polymer injected into the mold cavity half is selected from the group consisting of polystyrene, polyolefin, polycarbonate, polyvinyl chloride, polyurethane, and acrylic.

16. The method of claim 14, wherein the membrane comprises a polymeric microporous membrane.

17. A method for analyzing biomolecules comprising:
providing at least binding agent comprising one or more probe nucleic acids having nucleotide sequences on a first surface of a microporous membrane of an analysis device such that the probe nucleic acids are immobilized, the probe nucleic acid nucleotide sequences being complementary to a nucleotide sequence of one or more biomolecules of interest,
the analysis device comprising the microporous membrane integrally bonded to a non-porous polymeric injection-molded support, the membrane having a first surface for receiving the probe nucleic acids and one or more samples containing the biomolecule(s), the membrane having a second surface integrally bonded to a surface of the support;
depositing the sample(s) onto the first surface of the membrane such that the biomolecule(s) contact the probe nucleic acid(s) and one or more complexes are formed, each formed complex comprising a probe nucleic acid nucleotide sequence bound to the complementary nucleotide sequence of the biomolecule; and,
detecting the complexes.

18. The analysis device according to claim 2, wherein the membrane has a first and second surface and a bulk disposed between the first and second surface, the bulk having a thickness and wherein the bulk thickness is reduced by the heat and pressure of injection molding when the polymeric support is formed.

19. The analysis device according to claim 2, wherein the bulk thickness of the membrane is reduced by at least about ten percent when compared to the bulk thickness of the membrane before bonding it to the support.

20. The analysis device of claim 2, wherein the membrane comprises a sulfone membrane.

21. The analysis device of claim 2, wherein the membrane comprises a polyamide membrane.
